(19) United States
(12) Patent Application Publication
(10) Pub. No.: US 2015/0088231 A1
(43) Pub. Date: Mar. 26, 2015

(54) OCULAR TREATMENT SYSTEM AND METHOD USING RED AND GOLD PHOTOTHERAPY

(71) Applicant: CXL Ophhthalmics, LLC, Encinitas, CA (US)

(72) Inventors: Roy S. Rubinfeld, Bethesda, MD (US); Raymond A. Hartman, Carlsbad, CA (US); Sandy T. Feldman, Del Mar, CA (US)

(73) Assignee: CXL Ophthalmics, LLC, Encinitas, CA (US)

(21) Appl. No.: 14/387,193

(22) PCT Filed: Mar. 26, 2013
(86) PCT No.: PCT/US2013/033923

§ 371 Date: Sep. 22, 2014

Related U.S. Application Data
(60) Provisional application No. 61/616,774, filed on Mar. 28, 2012.

(51) Int. Cl. A61N 5/06

(52) U.S. Cl. CPC A61N 5/0613; A61N 2005/0663; A61N 2005/0661; A61N 2005/0651; A61N 2005/0654; A61N 2005/067
USPC 607/89; 607/88; 607/90

(57) ABSTRACT

Systems and methods of ophthalmologic or ocular phototherapy treatment are provided herein. In some embodiments, the systems and methods of ophthalmic or ocular phototherapy treatment comprise applying a treatment light beam in a predetermined wavelength or wavelength range corresponding to gold (yellow to orange), red, or at least part of both the gold and red visible light wavelength ranges to at least a portion of an eye. The treatment light beam may be applied to an entire eye. In some embodiments, the selected treatment area is irradiated with light in the selected gold and/or red wavelength range at a predetermined dose for a selected time period as a primary treatment in order to treat various eye conditions and reduce eye pain or discomfort, or help to promote eye wound healing following injury or surgery, or as a secondary treatment to augment eye treatment using light in different phototherapy treatment ranges.

### CROSS-REFERENCE

This application claims priority to U.S. Provisional Application No. 61/616,774, filed Mar. 28, 2012, which is incorporated herein by reference in its entirety.

### BACKGROUND

Collagen cross-linking is a parasurgical treatment for multiple ophthalmic disorders. In some cases, collagen cross-linking may also be combined with other treatments to improve corneal strength or optical refraction. Treatment methods include mini asymmetric radial keratotomy, corneal ring segment inserts, or topography-guided laser. Corrective lenses are normally required after these treatments, but with smaller, more normalized prescriptions. Increased corneal symmetry allows for more comfortable contact lens wear, often of daily disposable lenses. Collagen crosslinking limits deterioration of vision, increases unaided and uncorrected vision, and may reduce the need for corneal transplantation.

### SUMMARY

Embodiments described herein provide for an improved ocular phototherapy treatment system and method using light in gold, red, or gold to red wavelength ranges. The terms “gold” or “gold light” as used herein refer to light at wavelengths within the yellow to orange visible light spectrum.

Disclosed herein, in certain embodiments, is a method for ophthalmologic or ocular phototherapy treatment, comprising irradiating at least a part of an eye with a predetermined dose of gold light, red light, or a combination of gold and red light for a predetermined time period. In some embodiments, the method further comprises applying a photosensitizing agent to the part of the eye. In some embodiments, the photosensitizing agent comprises a riboflavin and a tear solution. In some embodiments, the method further comprises applying a photochemical treatment to the part of the eye. In some embodiments, the photochemical treatment comprises the application of UV light. In some embodiments, the gold light has a wavelength between about 560 nm to about 630 nm. In some embodiments, the red light has a wavelength between about 630 nm to about 830 nm. In some embodiments, the combination of gold and red light has a wavelength between about 560 nm to about 830 nm. In some embodiments, the predetermined dose is between about 2 J/cm² to about 6 J/cm². In some embodiments, the predetermined time period is from about 5 minutes to about 10 minutes. In some embodiments, the at least a part of the eye is irradiated at periodic intervals of hours or days.

Disclosed herein, in certain embodiments, is an ocular phototherapy system comprising: an illumination source configured to provide a light output in a predetermined wavelength range comprising gold, red, or gold to red light; and at least one optical treatment head connected to the light output of the illumination source, the at least one optical treatment head comprising an optical projector configured to direct a phototherapy light beam in the predetermined wavelength range onto a predetermined region of an eye of a patient. In some embodiments, the gold light has a wavelength between about 560 nm to about 630 nm. In some embodiments, the red light has a wavelength range between about 630 nm to about 830 nm. In some embodiments, the combination of the gold and red light has a wavelength between about 560 nm to about 830 nm. In some embodiments, the illumination source is a fluorescent lamp, a light emitting diode, a laser diode, or a phosphor lamp. In some embodiments, the fluorescent lamp comprises a gold phosphor having a spectrum in the range from about 500 nm to about 600 nm and peaks at about 540 nm and about 580 nm. In some embodiments, the system further comprises an adjustable mounting mechanism for supporting and positioning the at least one optical treatment head. In some embodiments, the system further comprises at least one liquid light guide for transmitting light from the light output to the at least one optical treatment head.

Disclosed herein, in some embodiments, is a method of ophthalmologic or ocular phototherapy treatment, comprising irradiating at least part of the eye with a light. In some embodiments, the light is in a range corresponding to gold light, red light or both gold and red light. In some embodiments, irradiating comprises a predetermined dose. In some embodiments, irradiating comprises a predetermined time period.

In some embodiments, the phototherapy is applied in conjunction with a photosensitizing agent. In some embodiments, the photosensitizing agent is riboflavin. In some embodiments, the phototherapy is applied in conjunction with a photosensitizing agent and an eye treatment solution. In some embodiments, the eye treatment solution comprises a tear solution. In some embodiments, the phototherapy is applied in conjunction with a solution. In some embodiments, the solution is a tear solution. In some embodiments, the solution does not comprise riboflavin. In some embodiments, the solution comprises riboflavin. In some embodiments, the phototherapy is applied in conjunction with one or more agents. In some embodiments, the photosensitizing agent, solution, tear solution, and/or agent is used before, during, or after one or more ocular treatments. In some embodiments, the one or more ocular treatments comprise a photochemical treatment with UV light or the like. In some embodiments, the photosensitizing agent, solution, tear solution, and/or agent is used before, during, or after surgery to help speed up healing, reduce pain, or to seal wounds. In some embodiments, gold, red or red-gold phototherapy is used as an independent or stand alone treatment system and method for various eye diseases, infections, and other conditions. In some embodiments, gold, red or red-gold phototherapy is used in combination with one or more photosensitizing agents, solutions, tear solutions, agents, or combinations thereof.

Further disclosed herein, in some embodiments, is an ocular phototherapy system or device. In some embodiments, the ocular phototherapy system or device comprises an illumination source configured to provide a light output in a predetermined wavelength range comprising gold, red, or gold to red light, and at least one optical treatment head connected to the light output from the illumination source and comprising an optical projection system configured to direct a phototherapy light beam in the selected wavelength range onto a predetermined region of a patient's eye.

In some embodiments, the wavelength range of the phototherapy light is between about 500 nm to about 850 nm. In some embodiments, the wavelength range of the phototherapy light is between about 520 nm to about 830 nm. In some embodiments, the wavelength range of the phototherapy light is between about 550 nm to about 750 nm. In some embodiments, the wavelength range of the phototherapy light is between about 550 nm to about 680 nm. In some embodiments, the wavelength range of the phototherapy light is between about 550 nm to about 850 nm. In some embodiments, the wavelength range of the phototherapy light is between about 560 nm to about 830 nm. In some embodiments, the wavelength range of the phototherapy light is between about 550 nm to about 750 nm. In some embodiments, the wavelength range of the phototherapy light is between about 560 nm to about 670 nm. In some embodiments, the wavelength range of the phototherapy light is between about 560 nm to about 660 nm. In some embodiments, the wavelength range of the phototherapy light is between about 560 nm to about 630 nm. In some embodiments, the wavelength range of the phototherapy light is between about 600 nm to about 900 nm. In some embodiments, the wavelength range of the phototherapy light is between about 600 nm to about 850 nm. In some embodiments, the wavelength range of the phototherapy light is between about 650 nm to about 850 nm. In some embodiments, the wavelength range of the phototherapy light is between about 660 nm to about 830 nm. In some embodiments, the wavelength range of the phototherapy light is between about 630 nm to about 830 nm.

In some embodiments, the wavelength of the phototherapy light is less than or equal to about 900 nm, 890 nm, 880 nm, 870 nm, 860 nm, 850 nm, 840 nm, 830 nm, 820 nm, 810 nm, 800 nm, 790 nm, 780 nm, 770 nm, 760 nm, 750 nm, 740 nm, 730 nm, 720 nm, 710 nm, 700 nm, 690 nm, 680 nm, 670 nm, 660 nm, 650 nm, 640 nm, 630 nm, 620 nm, 610 nm or 600 nm. In some embodiments, the wavelength of the phototherapy light is less than or equal to about 850 nm. In some embodiments, the wavelength of the phototherapy light is less than or equal to about 830 nm. In some embodiments, the wavelength of the phototherapy light is less than or equal to about 680 nm. In some embodiments, the wavelength of the phototherapy light is less than or equal to about 670 nm. In some embodiments, the wavelength of the phototherapy light is less than or equal to about 660 nm. In some embodiments, the wavelength of the phototherapy light is less than or equal to about 650 nm. In some embodiments, the wavelength of the phototherapy light is less than or equal to about 640 nm. In some embodiments, the wavelength of the phototherapy light is less than or equal to about 630 nm.

In some embodiments, the wavelength of the phototherapy light is at least about 450 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm or more. In some embodiments, the wavelength of the phototherapy light is at least about 510 nm. In some embodiments, the wavelength of the phototherapy light is at least about 520 nm. In some embodiments, the wavelength of the phototherapy light is at least about 530 nm. In some embodiments, the wavelength of the phototherapy light is at least about 540 nm. In some embodiments, the wavelength of the phototherapy light is at least about 550 nm. In some embodiments, the wavelength of the phototherapy light is at least about 610 nm. In some embodiments, the wavelength of the phototherapy light is at least about 620 nm. In some embodiments, the wavelength of the phototherapy light is at least about 630 nm. In some embodiments, the wavelength of the phototherapy light is at least about 640 nm. In some embodiments, the wavelength of the phototherapy light is at least about 650 nm. In some embodiments, the wavelength of the phototherapy light is at least about 660 nm. In some embodiments, the wavelength of the phototherapy light is at least about 670 nm.

In some embodiments, the phototherapy light is in the wavelength range from around 560 nm to 830 nm which includes both gold and red light. In other embodiments, the light is in the range from around 560 nm to 660 nm.

In some embodiments, the illumination source is any suitable illumination source. In some embodiments, the illumination source is a fluorescent lamp. In some embodiments, the fluorescent lamp comprises a gold phosphor. In some embodiments, the illumination source is a light emitting diode (LED) or laser diode is. In some embodiments, the illumination source is a phosphor lamp. In some embodiments, the phosphor lamp generates the red-gold light wavelengths.

In some embodiments, the illumination source has a spectrum in the range from around 500 nm to 660 nm. In some embodiments, the spectrum of the illumination source peaks at 540 nm and around 580 nm. In some embodiments, the spectrum of the illumination source is optimized for ocular phototherapy.

Further disclosed herein, in some embodiments, is an ocular phototherapy treatment method comprising applying a light beam in a predetermined wavelength range between about 560 nm to about 680 nm to at least a portion of eye for one or more time periods. In some embodiments, the one or more time periods are between about 1 minute to about 60 minutes. In some embodiments, the one or more time periods are between about 2 minutes to about 50 minutes. In some embodiments, the one or more time periods are between about 3 minutes to about 40 minutes. In some embodiments, the one or more time periods are between about 4 minutes to about 30 minutes. In some embodiments, the one or more time periods are between about 5 minutes to about 20 minutes. In some embodiments, the one or more time periods are between about 5 minutes to about 15 minutes. In some embodiments, the one or more time periods are between about 5 minutes to about 10 minutes. In some embodiments, the one or more time periods is less than or equal to 60 minutes, 55 minutes, 50 minutes, 45 minutes, 40 minutes, 35 minutes, 30 minutes, 25 minutes, 20 minutes, 15 minutes, 14 minutes, 13 minutes, 12 minutes, 11 minutes, 10 minutes, 9 minutes, 8 minutes, 7 minutes, 6 minutes, or 5 minutes. In some embodiments, the one or more time periods is less than or equal to 15 minutes. In some embodiments, the one or more time periods is less than or equal to 14 minutes. In some embodiments, the one or more time periods is less than or equal to 13 minutes. In some embodiments, the one or more time periods is less than or equal to 12 minutes. In some embodiments, the one or more time periods is less than or equal to 11 minutes. In some embodiments, the one or more time periods is less than or equal to 11 minutes. In some embodiments, the time period is greater than or equal to about 30 seconds, 1 minute, 1.5 minutes, 2 minutes, 2.5 minutes, 3 minutes, 3.5 minutes, 4 minutes, 4.5 minutes, 5 minutes, 5.5 minutes, 6 minutes, 6.5 minutes, 7 minutes, 7.5 minutes, 8 minutes, 8.5 minutes, 9 minutes, 9.5 minutes, or 10 minutes. In some embodiments, the time period is greater than or equal to about 1 minute. In some embodiments, the time period is greater than or equal to about 1.5 minutes. In some embodiments, the time period is greater than or equal to about 2 minutes. In some embodiments, the time period is greater than or equal to about 2.5 minutes. In some embodiments, the time period is greater than or equal to about 3 minutes. In some embodiments, the time period is greater than or equal to about 3.5 minutes. In some embodiments, the time period is greater than or equal to about 4 minutes. In some embodiments, the time period is greater than or equal to about 4.5 minutes. In some embodiments, the time period is greater than or equal to about 5 minutes. In some embodiments, applying the light beam comprises 2 time periods, 3 time periods, 4 time periods, 5 time periods, 6 time periods, 7 time periods, 8 time periods, 9 time periods, 10 time periods, 11 time periods, 12 time periods, 13 time periods, 14 time periods, 15 time periods, 20 time periods, 25 time periods, 30 time periods, 35 time periods, 40 time periods or more. In some embodiments, the one or more time periods comprise one or more intervals. In some embodiments, the one or more intervals comprise 1 time per day, 2 times per day, 3 times per day, 4 times per day, 5 times per day, 6 times per day, 7 times per day, 8 times per day, 9 times per day, 10 times per day, 11 times per day, 12 times per day, 13 times per day, 14 times per day, 15 times per day, 16 times per day, 17 times per day, 18 times per day, 19 times per day, 20 times per day, 21 times per day, 22 times per day, 22 times per day, 23 times per day, 24 times per day or more. In some embodiments, the one or more intervals comprise 1 time per week, 2 times per week, 3 times per week, 4 times per week, 5 times per week, 6 times per week, 7 times per week, 8 times per week, 9 times per week, 10 times per week, 11 times per week, 12 times per week, 13 times per week, 14 times per week, 15 times per week, 16 times per week, 17 times per week, 18 times per week, 19 times per week, 20 times per week, 21 times per week, 22 times per week, 22 times per week, 23 times per week, 24 times per week or more. In some embodiments, the one or more intervals comprise 1 time per month, 2 times per month, 3 times per month, 4 times per month, 5 times per month, 6 times per month, 7 times per month, 8 times per month, 9 times per month, 10 times per month, 11 times per month, 12 times per month, 13 times per month, 14 times per month, 15 times per month, 16 times per month, 17 times per month, 18 times per month, 19 times per month, 20 times per month, 21 times per month, 22 times per month, 22 times per month, 23 times per month, 24 times per month or more. In some embodiments, the one or more intervals comprises every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or more. In some embodiments, the one or more intervals comprises every 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days or more. In some embodiments, the one or more intervals comprises every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks or more. In some embodiments, the one or more intervals comprises every 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or more.

In some embodiments, the dose of the light beam is between about 0.01 J/cm² to 10 J/cm². In some embodiments, the dose of the light beam is between about 0.1 J/cm² to 9 J/cm². In some embodiments, the dose of the light beam is between about 1 J/cm² to 8 J/cm². In some embodiments, the dose of the light beam is between about 1.5 J/cm² to 7 J/cm². In some embodiments, the dose of the light beam is between about 2 J/cm² to 6.5 J/cm². In some embodiments, the dose of the light beam is between about 2 J/cm² to 6 J/cm². In some embodiments, the dose of the light beam is less than or equal to about 10 J/cm², 9.5 J/cm², 9 J/cm², 8.5 J/cm², 8 J/cm², 7.5 J/cm², 7 J/cm², 6.5 J/cm², 6.4 J/cm², 6.3 J/cm², 6.2 J/cm², 6.1 J/cm², 6 J/cm², 5.9 J/cm², 5.8 J/cm², 5.7 J/cm², 5.6 J/cm², or 5.5 J/cm². In some embodiments, the dose of the light beam is less than or equal to about 6.5 J/cm². In some embodiments, the dose of the light beam is less than or equal to about 6.3 J/cm². In some embodiments, the dose of the light beam is less than or equal to about 6.1 J/cm². In some embodiments, the dose of the light beam is less than or equal to about 6.0 J/cm². In some embodiments, the dose of the light beam is greater than or equal to about 0.01 J/cm², 0.05 J/cm², 0.1 J/cm², 0.15 J/cm², 0.2 J/cm², 0.3 J/cm², 0.4 J/cm², 0.5 J/cm², 0.6 J/cm², 0.7 J/cm², 0.8 J/cm², 0.9 J/cm², 1 J/cm², 1.2 J/cm², 1.4 J/cm², 1.5 J/cm², 1.6 J/cm², 1.7 J/cm², 1.8 J/cm², 1.9 J/cm², 2.0 J/cm², 2.1 J/cm², 2.2 J/cm², 2.3 J/cm², 2.4 J/cm², or 2.5 J/cm². In some embodiments, the dose of the light beam is greater than or equal to about 1.5 J/cm². In some embodiments, the dose of the light beam is greater than or equal to about 1.6 J/cm². In some embodiments, the dose of the light beam is greater than or equal to about 1.7 J/cm². In some embodiments, the dose of the light beam is greater than or equal to about 1.8 J/cm². In some embodiments, the dose of the light beam is greater than or equal to about 1.9 J/cm². In some embodiments, the dose of the light beam is greater than or equal to about 2.0 J/cm². In some embodiments, the light beam wavelength is in the range of 560 nm to 630 nm and the dose of the light beam is between about 2 J/cm² to about 6 J/cm².

In some embodiments, gold and red lights have advantageous therapeutic effects when applied to the eye separately or in combination, and assist in mitochondrial rejuvenation. In some embodiments, gold and red lights have synergistic effects when applied to the eye separately or in combination. In some embodiments, gold, red, or red-gold ocular phototherapy helps alleviate pain and discomfort following injury or eye surgery, in healing wounds following eye injury or surgery, as well as in treating eye conditions. In some embodiments, eye conditions include, but are not limited to, infections, glaucoma, pterygium, and dry eye.

Other features and advantages of the present disclosure will become more readily apparent to those of ordinary skill in the art after reviewing the following detailed description and accompanying drawings.

### BRIEF DESCRIPTION OF THE DRAWINGS

The details of the present disclosure, both as to its structure and operation, may be gleaned in part by study of the accompanying drawings, in which like reference numerals refer to like parts, and in which:

FIG. 1 is a perspective view of a stand-alone gold, red or red-gold ocular phototherapy treatment apparatus according to embodiments of the disclosure;

FIG. 2 is a cross-sectional view of the optical treatment heads of FIG. 1 according to embodiments of the disclosure;

FIG. 3 is a block diagram illustrating the optical source unit of FIG. 1;

FIG. 4 is a functional block diagram of an ocular treatment system using the apparatus of FIGS. 1 to 3 according to embodiments of the disclosure;

FIG. 5 is a spectrum of a suitable phosphor providing light wavelengths in the gold and red light range, which may be incorporated in a phosphor lamp in the optical source unit;

FIG. 6 is a cross-sectional view of a device comprising a multi-functional optical treatment head including a gold or red-gold phototherapy device according to embodiments of the disclosure;

FIG. 7 is a perspective view of a swivel mounting assembly for bilateral optical treatment heads each incorporating a gold or red-gold phototherapy device; and

FIG. 8 is a functional block diagram of a combined treatment system for cross-linking phototherapy incorporating one or more gold or gold/red light phototherapy devices.

### DETAILED DESCRIPTION

Certain embodiments as disclosed herein provide for ocular treatment systems and methods for various eye conditions using phototherapy with a light source emitting light in gold, red or both gold and red light wavelengths. In some embodiments, gold, red or red-gold light phototherapy is used to treat various eye conditions. In some embodiments, gold, red or red-gold phototherapy is used in conjunction with corneal strengthening or cross-linking treatment. In some embodiments, gold, red or red-gold phototherapy is used in conjunction with non-cross-linking applications. In some embodiments, gold, red or red-gold phototherapy is used to reduce eye pain or discomfort as a result of eye disorders, wounds, or surgery, assist wound healing after injury or eye surgery, and as therapy for oxidative conditions of the eye. In some embodiments, gold, red or red-gold phototherapy is used to treat glaucoma, macular degeneration, dry eye, and the like. In some embodiments, gold, red or red-gold phototherapy is used to treat or reduce inflammation or infection.

After reading this description it will become apparent to one skilled in the art how to implement the methods and devices of the disclosure in various alternative embodiments and alternative applications. However, although various embodiments of the present disclosure will be described herein, it is understood that these embodiments are presented by way of example only, and not limiting.

Gold light phototherapy as used herein refers to light in the yellow to orange wavelength range of the visible light spectrum, specifically around 560 nm to 630 nm. In some embodiments, the treatment wavelengths extend from the gold into the lower wavelengths of the visible red light range, for example 570 nm to 650 nm, or are confined to gold light wavelengths.

In some embodiments, any suitable light source is used to provide light in the desired gold or red-gold wavelength range, for example a fluorescent lamp with a suitable phosphor coating having a spectrum in the desired range, or a light emitting diode (LED) or laser diode. In some embodiments, illumination source is a fluorescent lamp. In some embodiments, the illumination source is a phosphor lamp. In some embodiments, the illumination source is an LED system. In some embodiments, the illumination source is a laser diode.

In some embodiments, the gold, red or red-gold light phototherapy device is provided in a stand alone gold, red or red-gold phototherapy system, or apparatus. In some embodiments, the gold, red or red-gold phototherapy device comprises a gold, red or red-gold phototherapy system incorporated into one or more eye treatment equipment. In some embodiments, the gold, red or red-gold phototherapy device is a multi-functional system. In some embodiments, the one or more eye treatment equipment comprise a UV photochemical treatment system. For example, a gold/red-gold light source is incorporated into a UV photochemical treatment system or other eye treatment system in alternative embodiments.

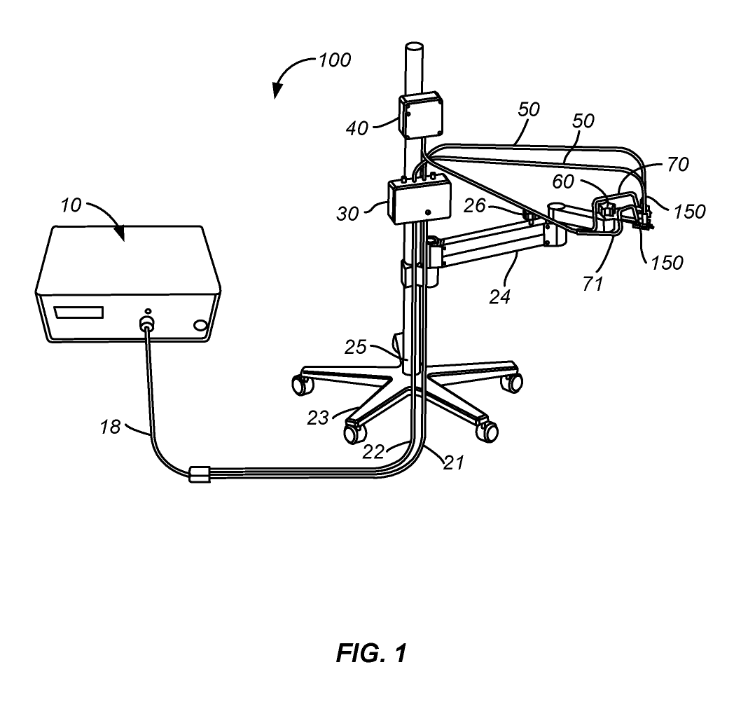

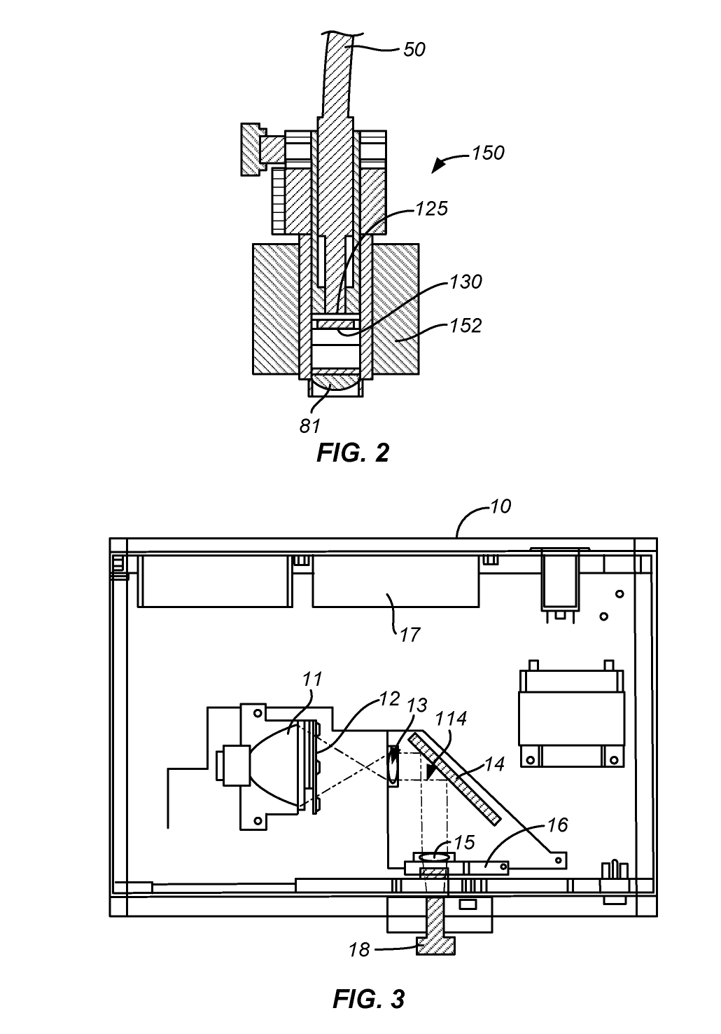

FIG. 1 to FIG. 4 illustrate an embodiment of a stand alone gold, red or red-gold phototherapy apparatus or system 100. As shown in FIG. 3, an illumination source unit 10 comprises a light source 11 that delivers light at a user-selected excitation wavelength to bifurcated, visible light transmissive liquid light guide 18. As shown in FIG. 1, the light guide 18 splits into separate light guide outputs 21 and 22 that are connected to illumination intensity adjustment module 30 mounted on a mobile pole stand comprised of pole 25 mounted on a base 23 with casters. In some embodiments, other support stands of different configuration are used in place of pole 25 with base 23. In some embodiments, outputs of module 30 are connected by light guides 50 to respective left and right optical treatment devices or heads 150. FIG. 2 depicts an example of an optical treatment device or head 150. In some embodiments, the light guides are optical fiber bundles. In some embodiments, the left and right optical treatment heads 150 are identical. In some embodiments, the dual optical treatment heads are replaced with a single treatment head connected to the light source unit 10 via a single light guide. In some embodiments, the phototherapy system does not comprise an intensity adjustment module 30 (FIG. 1). In some embodiments, the light guides 18, 21, 22 are connected directly to optical treatment heads 150, with filters for controlling light intensity or dosage as discussed in more detail below.

In some embodiments, the pole 25 allows attachment and vertical positioning of an adjustable mounting mechanism including articulating arm 24 on which the treatment heads 150 are mounted, and provides mounting points for illumination intensity adjustment module 30 and an optional optical monitoring module 40. The illumination source unit 10 is shown as separate from the mobile stand but is affixed to the stand in another embodiment. In some embodiments, the illumination source unit 10 is separated from the mobile stand pole. In some embodiments, the illumination source unit 10 is attached to the pole 25. In some embodiments, the optical treatment heads 150 are swivel mounted on the arm 24 for angular adjustment, and the separation between the heads 150 is adjustable to accommodate patients with different eye spacings. In some embodiments, light guides 18, 21, 22, and 50 which conduct the excitation energy to each optical treatment head are liquid light guides. Liquid light guides generally have greater transmission efficiency for visible light than optical fiber bundles while providing greater flexibility to allow for adjustment of the position of each treatment unit. In some embodiments, the liquid light guides homogenize light beams collected from non-homogeneous light sources or reflectors.

FIG. 3 illustrates the layout of the illumination source assembly with a phosphor lamp 11 incorporating a suitable phosphor for providing light in the desired wavelength range as the light source. In some embodiments, the lamp is multi-spectral and emits light in visible yellow and orange (“gold”) wavelength ranges as well as visible red wavelengths. A microprocessor 17 controls the opening and closing of shutter 12 that either blocks or allows passage of radiation emitted from the lamp. Shutter 12 is a mirrored aluminum material to reflect radiation away from the optical path. In some embodiments, the reflective quality of the material prevents a heat build up on the shutter and potential transfer of heat to the connecting solenoid assembly. In some embodiments, the shutter is affixed to a rotary solenoid to affect the opening and closing operation. In some embodiments, rotary solenoids are high reliability components with normal lifetimes exceeding 1 million cycles. In some embodiments, when shutter 12 is opened, the light from the lamp reflector is collected by collimating lens 13 and directed to mirror 14 that reflects light towards focusing lens 15 and into the input of bifurcated light guide 18. In some embodiments, an optional filter assembly 16 on a slide mechanism is connected to an actuating switch on the front panel, for allowing a user to switch between different wavelength ranges for treatment purposes, if desired. For example, in some embodiments, the filter assembly has filters having a 10 nm bandwidth at 580 nm, 595 nm, and 640 nm for selective placement in the light path into light guide 18.

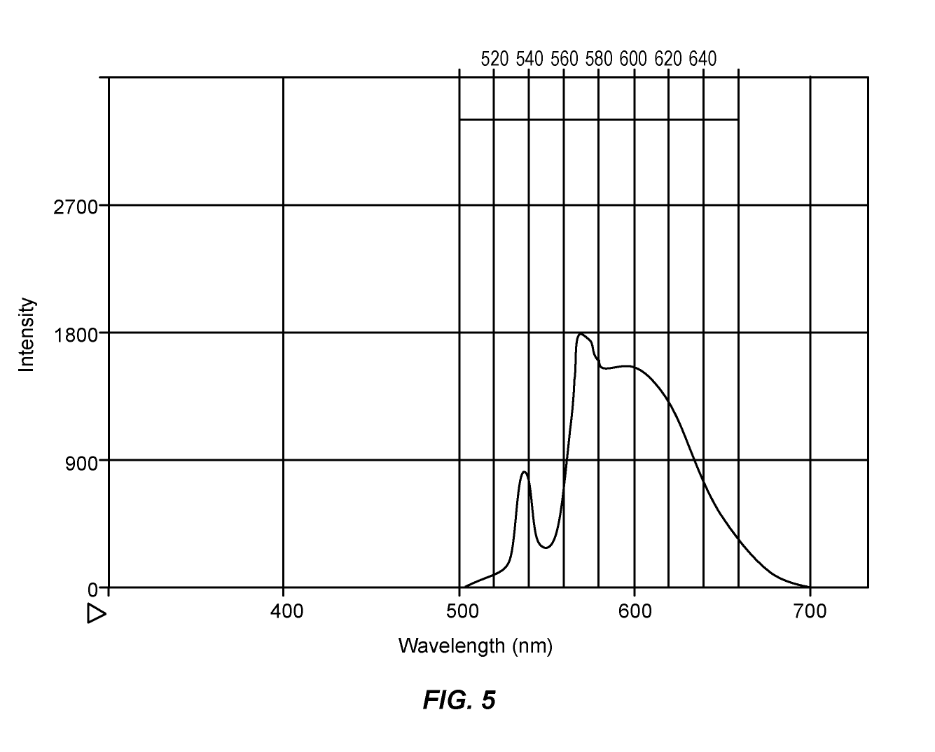

FIG. 5 illustrates a spectrum of one example of a gold or yellow-orange phosphor which provides wavelengths in proportions advantageous for red-gold light phototherapy. As illustrated, the spectrum extends from around 520 nm to 660 nm (including the entire wavelength range for yellow and orange visible light and extending into the lower wavelength ranges for visible red light), and has peaks at 540 nm and close to 580 nm. In some embodiments, the light spectrum peaks at between about 500 to 600 nm. In some embodiments, the light spectrum peaks at between about 510 to about 590 nm. In some embodiments, the light spectrum peaks at between about 520 to about 580 nm. In some embodiments, the light spectrum peaks at between about 530 to about 580 nm. In some embodiments, the light spectrum peaks at between about 540 to about 580 nm.

FIG. 2 illustrates the optical arrangement in one of the treatment heads 150 in more detail. Each optical treatment head 150 is mounted at the end of a support arm in holder 152, and incorporates an optical mask or reticule 130 for controlling the size and shape of the beam and a projection optic or lens 81 which is located at the exit end of the treatment head, as illustrated in FIG. 2. The exit end 125 of light guide 50 is secured in the treatment head and directs light in the selected gold or red-gold wavelength range onto optical mask 130. In some embodiments, mask 130 has a central circular opening and the mask and lens are configured to focus a circular spot of predetermined diameter on the eye at a predetermined working distance from the exit end of the optical treatment head. In some embodiments, different masks provide for different beam diameters in a suitable range to cover a normal range of eye dimensions, or different spot sizes and shapes for treatment of different regions of the eye, as described in more detail below. In some embodiments, a kit of different masks or slides with different aperture sizes, shapes and patterns is provided, including circular and non-circular apertures of various sizes, annular apertures, square or slit-shaped apertures, and the like.

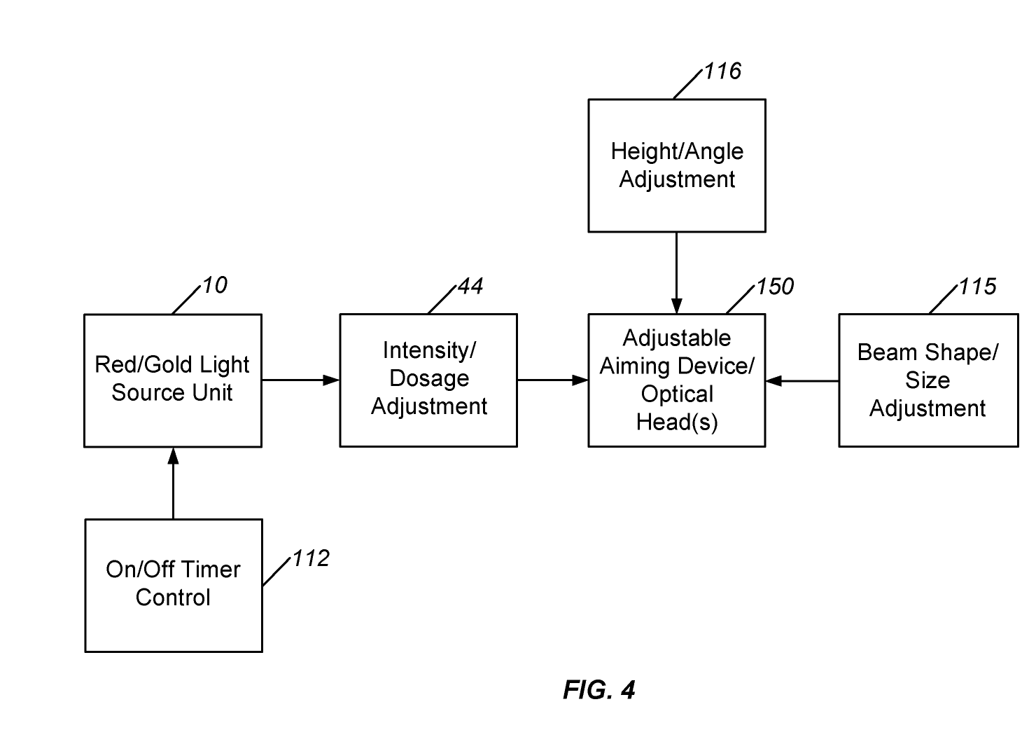

FIG. 4 depicts a functional block diagram of the various components of a treatment system using the apparatus of FIG. 1 to FIG. 3. In some embodiments, a computer controlled on-off timer 112 is connected to red-gold light source unit 10. In some embodiments, a light intensity/dosage adjustment device 44 is provided in the light path from unit 10 to optical head or heads 150, or is incorporated in an optical head 150. In some embodiments, the adjustment device comprises a holographic diffuser or attenuator filters incorporated in treatment heads 150. In some embodiments, beam shape and size adjustment 115 is provided by positioning selected masks or slides in the light path. Additionally, in some embodiments, a height and angle adjuster 116 for the optical heads 150 is provided. In some embodiments, adjustments are made manually or remotely using a suitable computer control system.

In some embodiments, the system illustrated in FIG. 1 to FIG. 4 is used in a stand-alone ocular treatment method. In some embodiments, the system illustrated in FIG. 1 to FIG. 4 is used in an ocular treatment method comprising medications or fluids applied to the eye. In some embodiments, the medications or fluids enhance the ocular treatment. In some embodiments, the medications or fluids comprise saturated tears or riboflavin solutions. In some embodiments, the ocular treatment method further comprises one or more ocular treatments or surgeries. In some embodiments, the treatment system is used in conjunction with one or more ocular treatments or surgeries. In some embodiments, the treatment system is used prior to one or more ocular treatments or surgeries. In some embodiments, the treatment system is used simultaneously with one or more ocular treatments or surgeries. In some embodiments, the treatment system is used after one or more ocular treatments or surgeries. In some embodiments, the system is incorporated with a corneal cross-linking system or method, for example the photochemical cross-linking system described in co-pending patent application Ser. No. 13/034,488 filed on Feb. 24, 2011, the entire contents of which are incorporated herein by reference. In other embodiments, gold, red or red-gold light ocular phototherapy is used in ocular treatment which does not involve cross-linking.

In some embodiments, gold, red-gold, and red light ocular phototherapy is beneficial in conjunction with photochemical cross-linking for treatment of keratoconus and ectasia, for example with photochemical cross-linking as described in co-pending patent application Ser. No. 13/034,488, referenced herein. In some embodiments, gold, red or red-gold ocular phototherapy issued for treating various other conditions in the eye without cross-linking. In some embodiments, gold light alone, red light alone, or both gold and red light wavelengths are used, at selected doses and for selected treatment times for non-cross-linking treatment of one or more ocular diseases, ocular infections, or for promoting wound healing and pain reduction following eye injuries, during or after refractive corneal surgery. Examples of ocular diseases include, but are not limited to, pterygium, glaucoma, and dry eye. Examples of refractive corneal surgery include, but are not limited to, PRK, LASIK, Intacs, conductive keratoplasty (CK), and other thermokeratoplasty treatments, lens based refractive surgery, retinal surgery or retinal or glaucoma laser surgery, intraocular surgery such as cataract surgery, as well as eyelid surgery. In some embodiments, phototherapy in gold, red-gold, and red light wavelengths is used to accelerate the healing process with refractive surgeries, by applying the phototherapy before, during and/or immediately after surgery. In some embodiments, gold, red or red-gold ocular phototherapy treatment is used at various times during the healing period following one or more ocular procedures. In some embodiments, gold, red or red-gold ocular phototherapy is used at various times during the healing period following PRK in which the epithelium is not healed. In some embodiments, gold, red-gold, or red light phototherapy is applied at various time intervals, for example daily or hourly, particularly early in the postoperative period. In some embodiments, gold, red or red-gold ocular phototherapy also has an effect on preserving nerve function despite interruption of nerves during refractive surgeries, such as PRK and LASIK, and helps to reduce dry eye.

In some embodiments, gold, red or red-gold ocular phototherapy assists in mitochondrial ATP production and thus resists cell damage or cell death. In some embodiments, ATP production is a key to survival of cells in the eye against cellular stress. In some embodiments, cytochrome C oxidase is a very specific enzyme in what is referred to as the Electron Transport Chain to production of ATP. In some embodiments, cytochrome C oxidase is the physical location where oxygen is “burned” in human metabolism. In some embodiments, oxygen molecules go to the Cu—Fe center(s) of this enzyme and provide the electromotive force for electron transport that creates ATP. In some embodiments, conditions or effects which act to block cytochrome C oxidase are the same as blocking the ability to use oxygen and cellular death ensues. In some embodiments, red light at around 670 nm “frees up” formic acid from the cytochrome c oxidase enzyme, thus allowing oxygen to access the Cu—Fe center and provide oxidative phosphorylation to make ATP. In some embodiments, cytochrome C oxidase has binuclear centers. In some embodiments, there are two sets of Cu—Fe pairs in cytochrome C oxidase. In some embodiments, one of these Cu—Fe pairs can absorb energy in red wavelengths from about 630 nm to about 830 nm. In some embodiments, absorption of red wavelengths from about 630 nm to about 830 nm results in changes in the physical configuration of cytochrome C oxidase, thus releasing the compound that is blocking the oxygen access to the enzyme. In some embodiments, the second center (called Cu(b)-Fe) does not absorb in the red light wavelength range. In some embodiments, the second center absorbs at a specific wavelength of 595 nm in the yellow to orange range (“gold” range). In some embodiments, by including both wavelengths (595 nm and 640 nm) in phototherapy, both sets of Cu—Fe pairs in the enzyme are unblocked, allowing more oxygen to get to the CCO and ATP production to start, reinvigorating cellular activity.

In some embodiments, the treatment or healing mechanism of gold, red or red-gold light relates in part to “overriding” some of the controls on mitochondrial ATP production. In some embodiments, gold, red or red-gold light ocular phototherapy is a useful treatment modality to overcome cellular “senescence” due to hypoxia or environmental factors, such as an environmental condition that stimulates the production of nitric oxide (for example rubbing the eyes, injury to the eyes, or the like). In some embodiments, gold, red, or red-gold ocular phototherapy stimulates, increases or modulates cell proliferation. In some embodiments, gold, red or red-gold light phototherapy is helpful in slowing the apoptotic progression, by modulating the natural “governor” for ATP production. In some embodiments, gold, red, or red-gold ocular phototherapy reduces, prevents, modulates, or slows apoptotic progression. In some embodiments, the mitochondria controls and stops ATP production via a number of signaling molecules that initiate activity on mitochondrial nitric oxide synthase (mtNOS). In some embodiments, mitochondria have a system to regulate ATP production through the production of nitric oxide. In some embodiments, nitric oxide is a competitive inhibitor of oxygen in the electron transport chain. In some embodiments, when nitric oxide is produced, the NO molecule “sits” in the space on an enzyme (cytochrome c oxidase or CCO) that is the normal “seat” for oxygen. In some embodiments, since oxygen is blocked by the NO molecule, no ATP production occurs. In some embodiments, nitric oxide is the mitochondrial energy brake. In some embodiments, as disclosed herein, red and/or gold light that strikes the CCO—NO complex photodissociates the NO away from the complex and allows oxygen to get to the CCO, so that ATP production starts. In some embodiments, the gold and/or red light induced photodissociation starts ATP production and thus reinvigorates cellular activity and healing. In some embodiments, ATP production (at the proper times) is the key to survival against cellular stress.

In some embodiments, in addition to blocking oxygen access to cytochrome c oxidase, nitric oxide also blocks the metal centers of other metalloenzymes like catalase and glutathione peroxidase, and the result is that these enzymes lose function. In some embodiments, when catalase and glutathione peroxidase lose function hydrogen peroxide builds up and kills the cells. Generally speaking, when nitric oxide levels are high as a result of inflammation or hypoxia, cells are lost because they don't have energy (nitric oxide blocking cytochrome c oxidase) or they can't disproportionate hydrogen peroxide to basal levels (oxidative death). In some embodiments, red-gold light phototherapy in the range of 595 nm to 680 nm doubles the capacity over red light/IR phototherapy (630 nm to 830 nm) for freeing up cytochrome c oxidase. In some embodiments, gold, red or red-gold phototherapy frees up catalase and glutathione peroxidase.

In some embodiments, gold, red, or red-gold phototherapy treatment as disclosed herein is in any selected range from around 560 nm to 750 nm. For example, a gold phototherapy range is from around 570 nm to 620 nm, a red-gold phototherapy range is from around 570 nm to 650 nm, and a red phototherapy range from around 620 nm to 680 nm. In some embodiments, gold and red light are applied to the eye successively. In some embodiments, gold and red light are applied to the eye simultaneously. In some embodiments, gold light alone is used. In some embodiments, for the cornea, an efficient depth of penetration is not a problem, and wavelengths from 570 nm to 650 nm have sufficient penetration. In some embodiments, shorter wavelengths have greater efficiency in dissociating the CCO—NO complex. In some embodiments, longer wavelengths present less burden to the eye due to the photopic response curve. In some embodiments, gold wavelength phototherapy in the range from 610 nm to 620 nm is beneficial for treating certain conditions or for reducing pain or discomfort. In some embodiments, the conditions or pain or discomfort are associated with an ocular disease, ocular infection, and/or ocular surgery or procedure.

In some embodiments, the 580 nm gold light wavelength is significant because it acts to photodissociate oxygen from hemoglobin and provide new localized oxygen to tissues. In some embodiments, photodissociation of oxygen from hemoglobin is important because red light phototherapy from 630 nm to 830 nm frees up nitric oxide from cytochrome c oxidase, but unless there is oxygen present to go to the Cu—Fe center for ATP production, there is no gain of energy. In some embodiments, the methods further comprise photodissociating oxygen from hemoglobin. In some embodiments, photodissociating oxygen from hemoglobin comprises applying gold light to at least a portion of the eye. In some embodiments, the method further comprises applying red light to at least a portion of the eye. In some embodiments, applying red light comprises stimulating ATP production. In some embodiments, nitric oxide inhibition of cytochrome c oxidase occurs when oxygen is low and nitric oxide is high, which will be the case during inflammation and hypoxia of wound healing. In some embodiments, oxygen enrichment of the area helps to prevent the displaced nitric oxide molecules from going back again to block the enzyme (the displacement is temporary). In some embodiments, displacing the nitric oxide with red light (630 nm to 830 nm) is generally helpful only when oxygen is present to initiate ATP production. In some embodiments, oxygen is available in vast amounts under skin tissue and behind the RPE of the retina, but it is bound to hemoglobin. In some embodiments, by tapping into the vast oxygen reservoir in the hemoglobin, the tissue can be re-oxygenated at 580 nm to allow or enhance ATP production. In some embodiments, oxygen is not displaced from hemoglobin by any wavelengths longer than 580 nm. In some embodiments, the oxygen photodissociation range is 550 nm to 580 nm, with 580 nm being selected because it has good depth penetration in the eye. In some embodiments, oxygen saturated tear solutions are added to the eye to enhance oxygen availability during gold or red-gold phototherapy. In some embodiments, the irradiation time period is important because the gold or red light only photodissociates nitric oxide (a competitive inhibitor of oxygen) during certain cycles of the electron transport chain. In some embodiments, the time for the electron transport chain to process electrons is about 10 minutes, so the time of phototherapy is extended to catch all of the various electron transport chains contained in the mitochondria during the appropriate part of the cycle. In some embodiments, the treatment time is appropriately controlled by the physician using these considerations. In some embodiments, gold or red light phototherapy, or a combination of red and gold phototherapy is delivered in a dose in the range of 2.0 J./cm² to 6.0 J./cm² for a three to seven minute period, but in other embodiments different time periods and doses are designed for various different treatment applications. In some embodiments, light at a dose of 4.5 J/cm² to 5.5 J/cm² is used for beneficial effects on ATP production. In other embodiments, a biphasic dose for exposed ocular cells of 3.0 J/cm^{2t} to 3.5 J/cm² over a 10 minute period is used. In some embodiments, the method comprises an irradiance of about 5 mw/cm² for the 10-minute period.

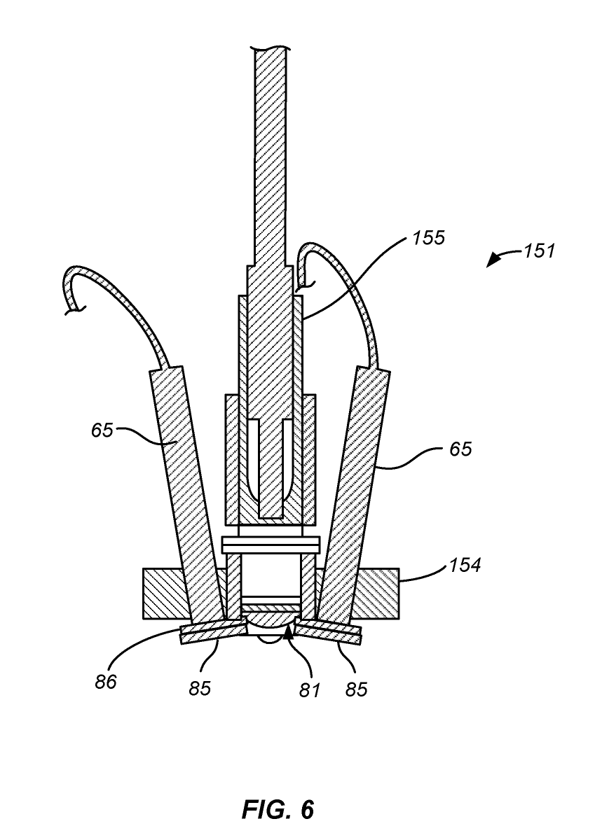

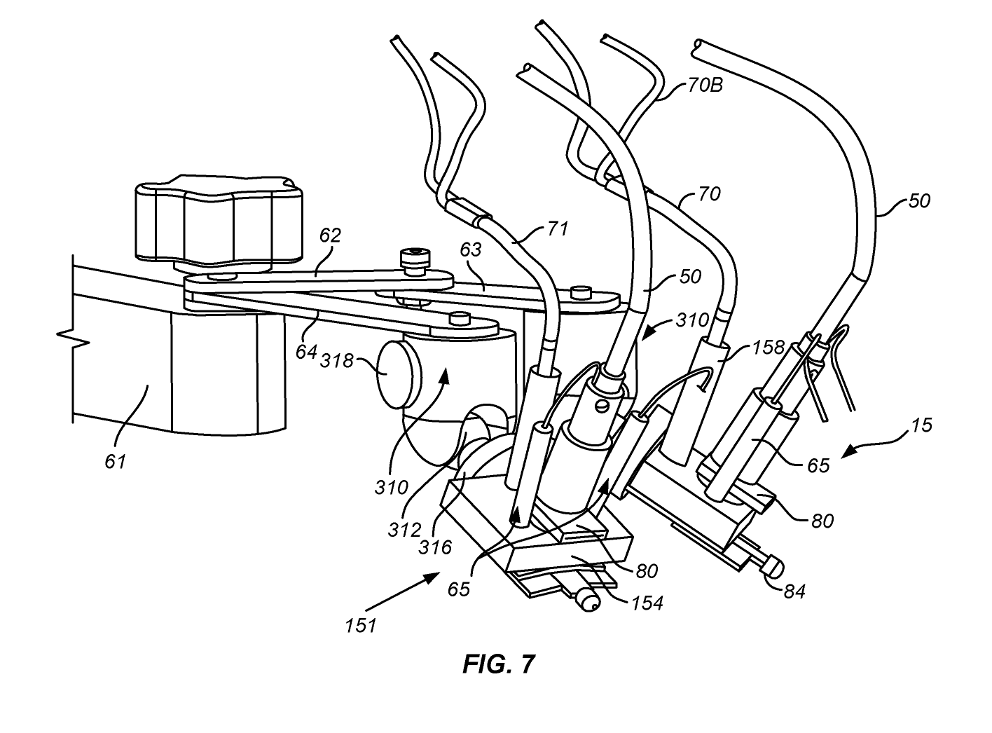

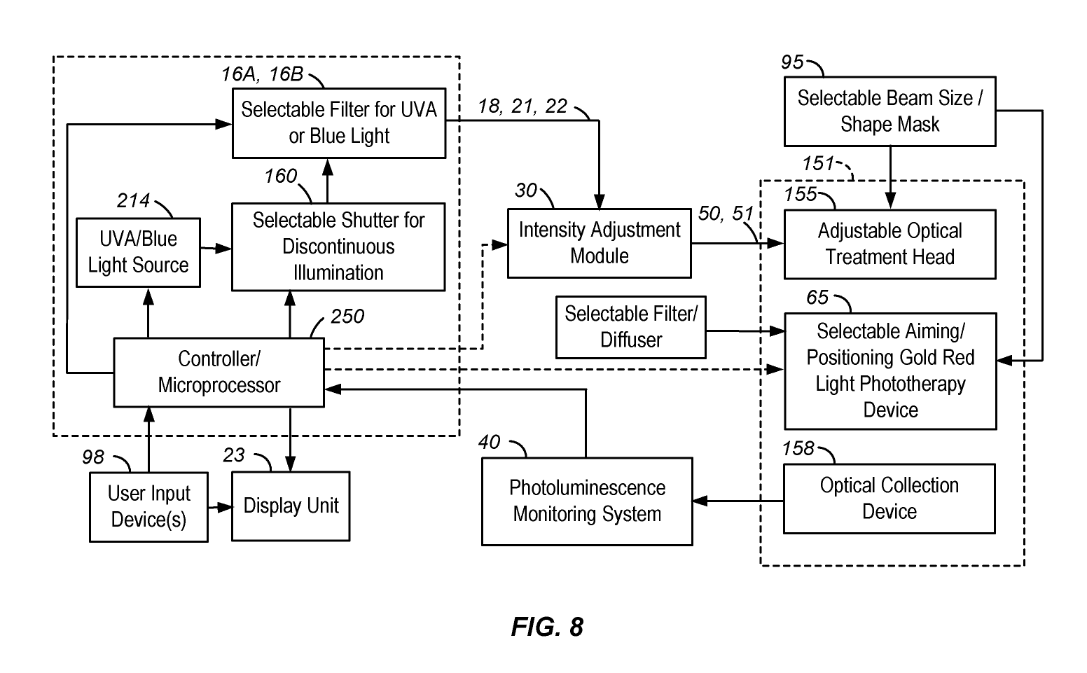

In some embodiments, a gold, red, or red-gold wavelength phototherapy device is provided in a multi-functional treatment device. In some embodiments, a gold, red, or red-gold phototherapy device is provided as an independent phototherapy device. FIG. 6 to FIG. 8 illustrate another embodiment in which one or more red-gold phototherapy treatment devices 65 are incorporated into a multi-functional treatment head 151, which are in some embodiments swivel mounted on a support arm 63 or 64 in a single or dual eye treatment apparatus. As illustrated in FIG. 6 and FIG. 7, each treatment head 151 includes a phototherapy treatment device 155, which is used in some embodiments for UV crosslinking treatment as described in co-pending application Ser. No. 13/034,488 referenced herein, a pair of angled red-gold phototherapy devices 65 and an optional optical collection device 158 which may be used for monitoring purposes.

In some embodiments, the treatment device 155, red-gold phototherapy devices 65 and optical collection device 158 are all mounted on a common support or mounting plate 154 with a swivel joint 310, 312, 316 connecting the mounting plate to the respective support arm 63 or 64. In some embodiments, phototherapy devices 65 also act as aiming or positioning devices to assist an operator in positioning the projection optic or lens 81 at a desired working distance from the cornea. In some embodiments, one or both devices 65 are light emitting diodes or laser diodes and are used both for gold or red-gold phototherapy applied before, after, or during phototherapy with treatment device 155, and for aiming or positioning purposes. Although the treatment device 155 in some embodiments is for corneal cross-linking phototherapy, other therapeutic devices are used in other embodiments. Additionally, in some embodiments, treatment device 155 is itself be a gold or red-gold phototherapy device as in FIG. 1 to FIG. 4.

In some embodiments, the distance of optic 81 from the cornea is determined to be equal to the desired working distance when the two aiming beams from laser diodes 65 coincide with each other as a single spot on the patient's eye. In some embodiments, if the aiming beams do not cross at the eye, the height adjustment knob on the articulating arm moves the optical heads up or down until the beams coincide at the correct position. In some embodiments, movement of the optical heads provides a more accurate method for positioning the optical heads at a predetermined distance relative to the patient's eyes. In some embodiments, the beams from the alignment laser diodes 65 are directed through selectable filters 85, 86 mounted on a mechanical slide 84 on the bottom of support 154, as illustrated in FIG. 7, so as to switch between aiming beams for optical head positioning purposes and gold, red-gold or red light beams for phototherapy purposes. In some embodiments, when the manual slide is pulled forward, filters 86 are placed in front of the output of laser diodes 65 and 66. In some embodiments, filters 86 are attenuating filters which reduce a nominal 3-mw diode output to a few hundred microwatts of output. In some embodiments, the beam shape remains unchanged. In some embodiments, the beam shape is a 3 mm dot. In some embodiments, the power is limited. In some embodiments, according to IEC60825, the maximum permissible exposure of a coherent beam from 400 nm to 700 nm on the eye is 0.002 J/cm². In some embodiments, the height alignment by the operator is completed in 3 or 4 seconds. In some embodiments, it is reasonable to design for a 80 second aiming beam alignment procedure. In some embodiments, a long aiming beam alignment procedure provides a safety factor for the patient. In some embodiments, a long aiming beam alignment procedure is greater than or equal to 30 seconds, 35 seconds, 40 seconds, 50 seconds, 55 seconds, 60 seconds, 65 seconds, 70 seconds, 75 seconds, 80 seconds, 85 seconds, 90 seconds, 95 seconds, or 100 seconds. In some embodiments, a 3-mw laser diode operated in pulse mode at a 3% duty cycle has a power output of 0.00009 W, and a 3 mm dot has an area of 0.071 cm². In some embodiments, in 80 seconds, a total of 0.142 J/cm² of non-attenuated light is delivered from both beams, so an attenuation filter with an optical density of 2 (99% reduction in energy) for filter 86 allows 80 seconds of laser beam alignment at a cumulative dose of 0.0014 J/cm². In some embodiments, when the mechanical slide 84 is moved to the back position, filters 85 are placed in front of the laser diodes. In some embodiments, filter 85 is a holographic circular light shaping diffuser with a 5 degree spread which shapes the laser diode beam to a circular area 10 mm to 12 mm in diameter at 3 inches, which is a suitable working distance from the eye. This provides laser diodes with a secondary use for providing gold, red-gold or red light phototherapy to ameliorate oxidative damage done to cells. In this case the holographic light shaping diffusers are 95% efficient in transmission and the laser diodes can be operated at full power giving about 5 mw/cm² for the combined beams over the 10 mm circle, thus providing a therapeutic dose of 3.0 J/cm² in a 10 minute period.

FIG. 8 illustrates an embodiment of a control system for a multi-function treatment device as illustrated in FIG. 6 and FIG. 7. In some embodiments, a controller or microprocessor 250 having a user control input 98 and an output display unit 23 is connected to a UVA/Blue light source 214 as well as a selectable shutter 160 for discontinuous illumination, and a selectable filter 16A, 16B for controlling the phototherapy treatment range. In some embodiments, light guides 18, 21, 22 extend from the light source output via the intensity adjustment module to the optical treatment device 155 in multi-function treatment head 151. In some embodiments, an optional optical collection device 158 is connected via photoluminescence monitoring system 40 to the controller 250. In some embodiments, the phototherapy system comprises a collection and monitoring arrangement. The collection and monitoring arrangement is described in detail in co-pending application Ser. No. 13/034,488 referenced herein, and is therefore not further described here.

In some embodiments, the selectable positioning device and red-gold phototherapy devices 65 are also connected to the output of controller 250 in order to move diffuser filter 85 into position when the devices are to be used for gold, red-gold, or red light phototherapy and to move attenuator filter 86 into position when the devices are to be used for positioning the head at the appropriate distance from the eye, whether for UV phototherapy or for red-gold phototherapy. In some embodiments, masks or slides 95 of different beam sizes and shapes may also be positioned in the light path from the optical treatment device 155 and in the light path from one or both of the red-gold phototherapy devices.

As noted herein, in some embodiments, the UVA/Blue light devices in FIG. 6 and FIG. 7 are omitted in an alternative embodiment, and in this alternative, the main optical treatment head 155 in FIG. 6 and FIG. 7 is a gold or red-gold phototherapy treatment head which receives light input from a phosphor lamp and light guide as in FIG. 1 to FIG. 5, or from a light emitting diode or laser diode which emits light in the desired frequency range. In some embodiments, the aiming/positioning devices are omitted altogether or white light devices are used for positioning the gold/red-gold phototherapy head at the appropriate distance from the eye.

In some embodiments, the phototherapy treatment system disclosed herein is a monocular, with a single optical treatment unit comprising an optical treatment head for directing a gold, red-gold, or red light beam into the eye, or bilateral, with two optical treatment units adjustably mounted on a support stand for treatment of both eyes simultaneously. In some embodiments, the optical treatment unit is limited to focusing a gold, red-gold or red light beam on a patient's eye or both eyes, or incorporates additional treatment or monitoring devices, such as the UVA-Blue light treatment device of FIG. 8. Where the system is bilateral, optical treatment units is, in some embodiments, identical. In some embodiments, the bilateral, optical treatment units are different. In some embodiments, the bilateral, optical treatment units are separately mounted to allow for adjusting the separation between the units. In further embodiments, more than two treatment units are provided. In some embodiments, the optical treatment unit mount allows for angular adjustment as well as for adjustment of separation between the optical treatment heads and distance from the eye, to allow for angular variations in the angle at which the light beam is directed into the eye, as well as distance variations of the treatment beams. In some embodiments, the light guide from the optical source unit is bifurcated to provide two separate light guide portions which direct light into the respective optical heads. In some embodiments, selective gold, red-gold, or red light irradiation patterns are provided by the use of pre-prepared reticules printed onto polyester plastic. These prepared reticules have apertures providing a variety of different light distribution patterns and sizes desired by the physicians. In some embodiments, an intensity adjustment device is provided at any suitable point between the light source and optical treatment head for adjusting the intensity or irradiance of the light beam emitted by each treatment head independently. In some embodiments, the system also provides for physician selection of continuous or discontinuous illumination, as well as selection of the on-off time period for discontinuous illumination or fractionation.

In some embodiments, gold, red-gold or red light phototherapy is applied to the eye before, during, at different times during, or after corneal strengthening treatment, surgical treatment, or other types of eye treatment. In some embodiments, gold, red or red-gold phototherapy reduces the effects of collateral oxidative damage to the eye caused by UVA/blue light in corneal cross linking treatment and/or unwanted reactive oxygen species (ROS) from photosensitizers such as riboflavin. In some embodiments, the reactive oxygen species is hydrogen peroxide. In some embodiments, this protective effect is accomplished by one or more gold, red-gold, or red light phototherapies that are employed at different times. In some embodiments, if applied before the ocular surgery or procedure, the phototherapy builds up anti-apoptotic stores of ATP. In some embodiments, if applied during the photochemical therapy, the light reduces the intracellular superoxide anion production in the mitochondria.

In some embodiments, if used post-procedure or after eye injury to accelerate the wound healing response, the effect of gold, red-gold or red light is anti-apoptotic for cells exposed to oxidative stress and may accelerate wound healing. In some embodiments, gold, red-gold or red light treatment is used for reduction of pain in corneal procedures and in other ocular surgery procedures, such as photorefractive keratectomy (PRK), cataract surgery, glaucoma surgery, and the like. In some embodiments, retinal diseases such as macular degeneration (a major and serious cause of vision loss), glaucoma, and others are treatable with light in the gold to red wavelength ranges. In some embodiments, the light source is a phosphor lamp having a suitable gold light emitting phosphor, or a red-gold light emitting diode combined with a holographic diffuser to control dosage, and is mounted in the optical treatment head or connected to the treatment head by a suitable light guide or guides.

The description of the systems and methods are provided to enable any person skilled in the art to make or use the systems, methods, and devices disclosed herein. Various modifications to these embodiments will be readily apparent to those skilled in the art, and the generic principles defined herein can be applied to other embodiments without departing from the spirit or scope of the disclosure. Thus, the disclosure is not intended to be limited to the embodiments shown herein but is to be accorded the widest scope consistent with the principles and novel features disclosed herein.

We claim:

1. A method for ophthalmologic or ocular phototherapy treatment, comprising irradiating at least a part of an eye with a predetermined dose of gold light, red light, or a combination of gold and red light for a predetermined time period.

2. The method of claim 1, further comprising applying a photosensitizing agent to the part of the eye.

3. The method of claim 2, wherein the photosensitizing agent comprises a riboflavin and a tear solution.

4. The method of claim 1, further comprising applying a photochemical treatment to the part of the eye.

5. The method of claim 4, wherein the photochemical treatment comprises the application of UV light.

6. The method of claim 1, wherein the gold light has a wavelength between about 560 nm to about 630 nm.

7. The method of claim 1, wherein the red light has a wavelength between about 630 nm to about 830 nm.

8. The method of claim 1, wherein the combination of gold and red light has a wavelength between about 560 nm to about 830 nm.

9. The method of claim 1, wherein the predetermined dose is between about 2 J/cm² to about 6 J/cm².

10. The method of claim 1, wherein the predetermined time period is from about 5 minutes to about 10 minutes.

11. The method of claim 1, wherein the at least a part of the eye is irradiated at periodic intervals of hours or days.

12. An ocular phototherapy system comprising:
(a) an illumination source configured to provide a light output in a predetermined wavelength range comprising gold, red, or gold to red light; and
(b) at least one optical treatment head connected to the light output of the illumination source, the at least one optical treatment head comprising an optical projector configured to direct a phototherapy light beam in the predetermined wavelength range onto a predetermined region of an eye of a patient.

13. The system of claim 12, wherein the gold light has a wavelength between about 560 nm to about 630 nm.

14. The system of claim 12, wherein the red light has a wavelength range between about 630 nm to about 830 nm.

15. The system of claim 12, wherein the combination of the gold and red light has a wavelength between about 560 nm to about 830 nm.

16. The system of claim 12, wherein the illumination source is a fluorescent lamp, a light emitting diode, a laser diode, or a phosphor lamp.

17. The system of claim 16, wherein the fluorescent lamp comprises a gold phosphor having a spectrum in the range from about 500 nm to about 600 nm and peaks at about 540 nm and about 580 nm.

18. The system of claim 12, further comprising an adjustable mounting mechanism for supporting and positioning the at least one optical treatment head.

19. The system of claim 12, further comprising at least one liquid light guide for transmitting light from the light output to the at least one optical treatment head.
